(19) United States
(12) Patent Application Publication
(10) Pub. No.: US 2010/0217371 A1
(43) Pub. Date: Aug. 26, 2010

(54) Device, System, and Method for Aiding Stent Valve Deployment

(75) Inventors: Michael Noone, Londonderry, NH (US); Alexander Hill, Blaine, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/393,385

(22) Filed: Feb. 26, 2009

(51) Int. Cl. A61F 2/84; A61F 2/24

(52) U.S. Cl. 623/1.11; 623/2.11

(57) ABSTRACT

A system for aiding implantation of a stented valve includes a delivery device and an outer balloon carried upon the delivery device. Additionally, the system includes a first inner balloon positioned within the outer balloon, a second inner balloon positioned within the outer balloon and adjoining the first inner balloon, and at least a first controller operable to introduce and remove fluid from each of the first inner balloon and second inner balloon.

### TECHNICAL FIELD

This invention relates generally to medical devices and particularly to a device, system, and method for aiding deployment of a stent valve.

### BACKGROUND OF THE INVENTION

Heart valves, such as the mitral and tricuspid valves, are sometimes damaged by diseases or by aging, which can cause problems with the proper function of the valve. The mitral and tricuspid valves consist of leaflets attached to a fibrous ring or annulus. In a healthy heart, the mitral valve leaflets overlap during contraction of the left ventricle, or systole, and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

One repair procedure involves implanting a stented valve through the mitral valve. The stented valve is aligned with the valve annulus and then fixedly attached to the valve annulus. The valve generally assists in reducing regurgitation, and providing improved valve closure during systole, while the stent assists in fixation and maintaining the position of the stented valve.

Implantation of the stented valve presents challenges based on the typical stent deployment techniques, including self-expanding stents and balloon expanding stents. Both techniques are best utilized within a substantially cylindrical structure, such as a blood vessel. However, in a non-cylindrical structure, such as a cardiac valve, these techniques can result in over-expansion along one axis, and under-expansion along another axis.

Therefore, it would be desirable to provide a device, system, and method for aiding stent valve deployment to overcome the aforementioned and other disadvantages.

### SUMMARY OF THE INVENTION

One aspect of the present invention is a system for aiding implantation of a stented valve. The system includes a delivery device and an outer balloon carried upon the delivery device. Additionally, the system includes a first inner balloon positioned within the outer balloon, a second inner balloon positioned within the outer balloon and adjoining the first inner balloon, and at least a first controller operable to introduce and remove fluid from each of the first inner balloon and second inner balloon.

Another aspect of the present invention is a method for aiding implantation of a stented valve. The method includes delivering a stented valve to a location near a heart valve, extending the stented valve through the heart valve, and inflating a first inner balloon and a second inner balloon within an outer balloon to expand the outer balloon to define at least a first major axis and a first minor axis, wherein the first major axis and first minor axis are not equally sized. The method further includes expanding the stented valve based on the inflation to implant the stented valve within the heart valve.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is a longitudinal cross sectional illustration of a distal portion of a system for aiding stent valve deployment, in accordance with the present invention;

FIG. 2 is a cross section of the distal portion of the system of FIG. 1 taken along line A-A, prior to expansion of the first inner balloon and second inner balloon;

FIG. 3 is a cross section of the distal portion of the system of FIG. 1 taken along line A-A, taken after expansion of the first inner balloon and second inner balloon;

FIG. 4 is a cross section of the distal portion of the system of FIG. 1 after expansion of the first inner balloon and second inner balloon; and

FIG. 5 is a cross section of a system of a system for aiding stent valve deployment, in accordance with the present invention.

Similar reference numbers are used throughout the drawings to refer to similar parts.

### DETAILED DESCRIPTION OF THE PRESENTLY PREFERRED EMBODIMENTS

One aspect of the present invention is a device having a bidirectional dilation balloon for aiding in implantation of a stented valve. An outer balloon has a first inner balloon and a second inner balloon positioned within the outer balloon so that when the first inner balloon and second inner balloon are inflated and deflated in series with inflating the outer balloon, the stented valve assumes a shape having a first major axis and a first minor axis.

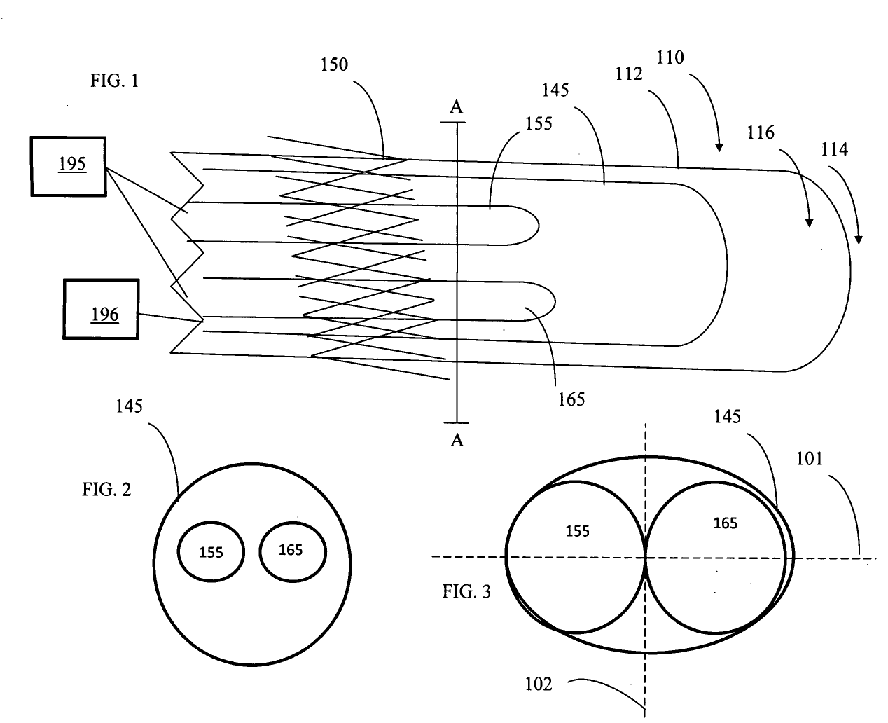

FIG. 1 illustrates system 10 for aiding in stent valve deployment. System 10 includes delivery device 110 having lumen 116. System 10 further includes a stented valve 150 to be delivered to a deployment site. Delivery device 110 includes body 112 shown with rounded distal tip 114. Delivery device 110 may have a straight tip or a preformed or deflectable distal tip that is capable of assuming a desired bend with respect to the longitudinal axis of the catheter to aid in delivering stented valve 150. The delivery device 110 can be any appropriate delivery device, such as a catheter or trocar. In embodiments using a catheter, the catheter can be a rapid exchange catheter or any other appropriate type. In one embodiment, distal tip 114 comprises a preset curve, e.g., a pigtail-shaped tip as such curves are known in the catheter art.

Body 112 comprises one or more flexible, biocompatible polymeric materials such as polyurethane, polyethylene, polyamide, fluoropolymers such as fluorinated ethylene propylene (FEP) or polytetrafluoroethylene (PTFE), or polyether-block amide (PEBA) co-polymer. Body 112 is sufficiently flexible to navigate the vasculature from an entry site to a location within the heart.

Delivery device 110 carries an outer balloon 145 and a first inner balloon 155 and a second inner balloon 165. Each of first inner balloon 155 and a second inner balloon 165 are positioned within the outer balloon 145, and each of first inner balloon 155 and a second inner balloon 165 are connected to a controller 195 which introduces and removes fluid to expand and contract the inner balloons. In one embodiment, the first inner balloon 155 and a second inner balloon 165 are disposed adjacent each other. In another embodiment, the first inner balloon 155 and a second inner balloon 165 are disposed on opposing sides of a barrier, such as a rigid device such as a wire, or a spacing device. In one embodiment, system 100 further includes a second controller 196 configured to introduce and remove fluid from the outer balloon. The second controller allows the inflation of the first inner balloon and second inner balloon to occur in series, or sequentially, with the inflation of the outer balloon. The inflation of the inner balloons (first inner balloon and second inner balloon) can be substantially simultaneous, and can occur either before the inflation and deflation of the outer balloon, or after the inflation and deflation of the outer balloon. In one embodiment, there are no stents or stented structures carried within the outer balloon 145.

Inflating each of the of first inner balloon 155 and a second inner balloon 165 results in an oblong shaped outer balloon, featuring a first major axis 101 (FIG. 3) and a first minor axis 102 (FIG. 3), such that the first major axis and first minor axis do not have the same length. The inflation results from introducing a volume of fluid into each balloon by the controller. In one embodiment, the inflation of the first inner balloon 155 and a second inner balloon 165 is substantially simultaneous. In another embodiment, the first inner balloon 155 and a second inner balloon 165 are inflated sequentially. In another embodiment, the first inner balloon 155 and a second inner balloon 165 are inflated to approximately the same size, which in other embodiments, the first inner balloon 155 and a second inner balloon 165 are inflated to different sizes, forming a shape which is larger at one end than the other end. Such an embodiment may be preferred in patients with oddly shaped and/or diseased valve pathways. For example, each of the first inner balloon and second inner balloon can be 8 mm balloons, or the first inner balloon can be an 8 mm balloon, while the second inner balloon is a 4 mm balloon. Balloons of other dimensions are contemplated.

Based on the inflation of the first inner balloon 155 and a second inner balloon 165, the stented valve 150 is deployed. In valves that feature axes of different lengths, such as the mitral valve, aortic valve or tricuspid valve, the use of the first inner balloon 155 and a second inner balloon 165 results in an expanded stent that more closely adheres to the shape of the anatomical valve. Additionally, more than two inner balloons can be utilized to achieve different deployment shapes such as a more triangular shape with three inner balloons (FIG. 5), or a quadrilateral arrangement with four inner balloons. As more inner balloons are included, however, the resulting shape will tend toward a circular shape, so there are limited benefits from increasing the number of balloons.

In the case of a stented valve replacement in the heart, any appropriate approach can be used, including a femoral, a trans-apical, trans-atrial, or a trans-septal route.

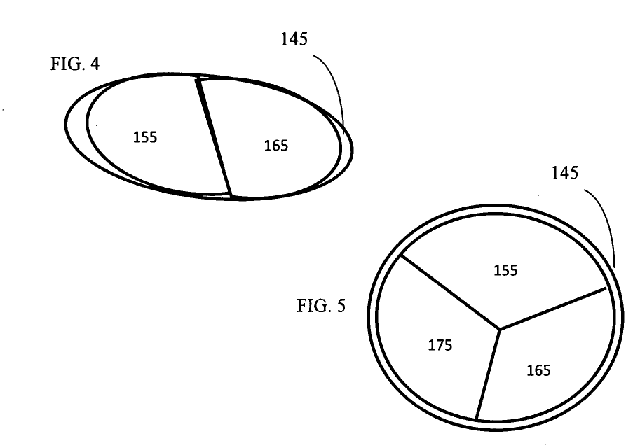

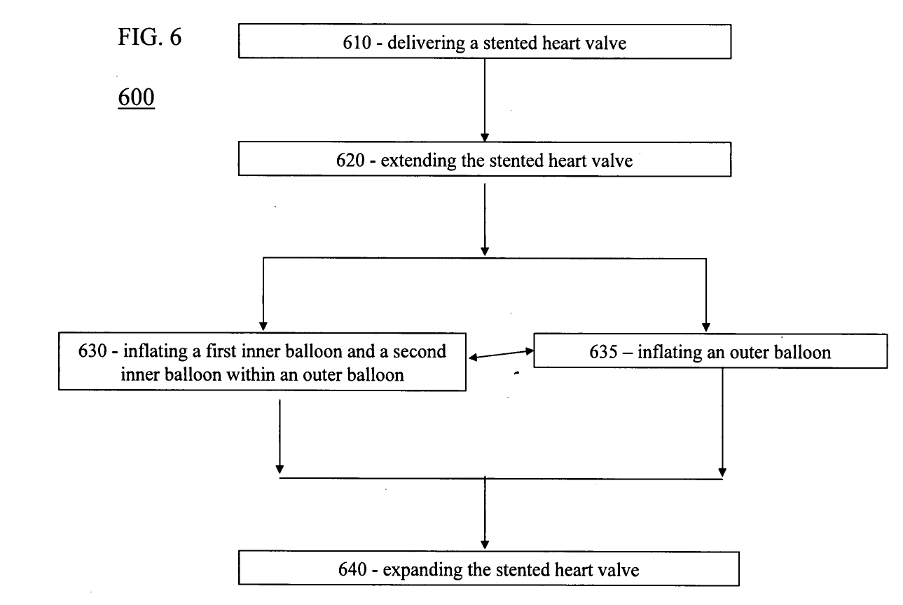

FIG. 2 illustrates a cross section of delivery device 110 at line A-A, in accordance with one aspect of the invention. In FIG. 2, the outer balloon 145 is illustrated in an expanded, or inflated position, while the first inner balloon 155 and second inner balloon 165 are illustrated substantially deflated. In contrast, FIG. 3 illustrates a cross section of delivery device 110 at line A-A while the outer balloon is substantially deflated, and while the first inner balloon 155 and second inner balloon 165 are illustrated substantially inflated. As illustrated in FIG. 3, the first inner balloon 155 and second inner balloon 165 maintain their idealized generally circular cross section, although in practice the actual cross section would more closely approximate the cross section in FIG. 4, as the sides of each of the first inner balloon 155 and second inner balloon 165 would deform on contacting the other balloon.

FIG. 6 illustrates a method 600 for aiding implantation of a stented valve in accordance with one aspect of the invention. Method 600 begins at 610 by delivering a stented valve to a location near a heart valve, and continues at step 620 by extending the stented valve through the heart valve. At step 630, a first inner balloon and a second inner balloon are inflated within an outer balloon. At step 635, an outer balloon is inflated. The inflation of the first inner balloon, second inner balloon, and outer balloon results in defining at least a first major axis and a first minor axis, wherein the first major axis and first minor axis are not equally sized. Steps 630 and 635 can be performed in either order. In other words, the first major axis can be formed either before forming the first minor axis (such as by inflating the first inner balloon and second inner balloon first), or after forming the first minor axis (such as when inflating the outer balloon first). At step 640, the stented valve is expanded based on the inflation of the first inner balloon, second inner balloon, and outer balloon to implant the stented valve within the heart valve.

The terms “distal” and “proximal” are used herein with reference to the treating clinician during deployment of the device; “Distal” indicates an apparatus portion distant from, or a direction away from the clinician and “proximal” indicates an apparatus portion near to, or a direction towards the clinician. However, those with skill in the art will recognize that the teachings of the invention may also be deployed at other cardiac valves or other locations in the body and may be used to insert stented valves in other openings or other structures within the body.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for aiding implantation of a stented valve, the system comprising:
a delivery device;
an outer balloon carried upon the delivery device;
a first inner balloon positioned within the outer balloon;
a second inner balloon positioned within the outer balloon and adjoining the first inner balloon;
at least one stented valve carried upon the outer balloon, the stented valve operable to assume an expanded position responsive to expansion of the first inner balloon, second inner balloon, and outer balloon; and
at least a first controller operable to introduce and remove fluid from each of the first inner balloon and second inner balloon.

2. The system of claim 1 wherein introducing a fluid into each of the first inner balloon and second inner balloon expands each of the first inner balloon and second inner balloon.

3. The system of claim 2 wherein expansion of each of the first inner balloon and second inner balloon expands an outer balloon that defines at least a first major axis and a first minor axis, wherein the first major axis and first minor axis are not equally sized.

4. The system of claim 1 wherein the controller is operable to inflate the first inner balloon and second inner balloon substantially simultaneously.

5. The system of claim 1 wherein the delivery device is one of a delivery catheter and a trocar.

6. The system of claim 1 further comprising a second controller operable to introduce and remove fluid from the first outer balloon.

7. The system of claim 6 wherein the first controller inflates the first inner balloon and second inner balloon in series with the second controller inflating the outer balloon.

8. The system of claim 1 wherein no stent is disposed within the outer balloon.

9. A method for aiding implantation of a stented valve, the method comprising:
delivering a stented valve to a location near a heart valve;
extending the stented valve through the heart valve;
inflating a first inner balloon and a second inner balloon within an outer balloon to expand the outer balloon to define at least a first major axis and a first minor axis, wherein the first major axis and first minor axis are not equally sized; and
expanding the stented valve based on the inflation of the first inner balloon and second outer balloon to implant the stented valve within the heart valve.

10. The method of claim 9 wherein inflating the first inner balloon and second inner balloon comprises inflating the first inner balloon and second inner balloon substantially simultaneously.

11. The method of claim 10 further comprising:
deflating the first inner balloon and second inner balloon; and
inflating the outer balloon based on the deflation of the first inner balloon and second inner balloon, and wherein expanding the stented valve is further based on the inflation of the outer balloon.

12. A method for aiding implantation of a stented valve, the method comprising:
delivering a stented valve to a location near a heart valve;
extending the stented valve through the heart valve;
inflating an outer balloon based on the extension;
deflating the outer balloon based on the inflation;
inflating a first inner balloon and a second inner balloon within the outer balloon to expand the outer balloon to define at least a first major axis and a first minor axis, wherein the first major axis and first minor axis are not equally sized; and
expanding the stented valve based on the inflation of the outer balloon, first inner balloon, and second inner balloon to implant the stented valve within the heart valve.

13. The method of claim 12 wherein inflating the first inner balloon and second inner balloon comprises inflating the first inner balloon and second inner balloon substantially simultaneously.

14. The method of claim 13 further comprising:
deflating the first inner balloon and second inner balloon; and
inflating the outer balloon based on the deflation of the first inner balloon and second inner balloon.
